(19) United States
(12) Patent Application Publication
(10) Pub. No.: US 2026/0201298 A1
(43) Pub. Date: Jul. 16, 2026

(54) CELL PRODUCTION DEVICE, VESSEL, AND SINGLE-USE KIT

(71) Applicant: Panasonic Intellectual Property Management Co., Ltd., Osaka (JP)

(72) Inventors: Hayase MINOURA, Gifu (JP); Naoshi YAMAGUCHI, Osaka (JP); Satoshi UEHARA, Kyoto (JP); Kazuaki NISHIO, Osaka (JP)

(21) Appl. No.: 19/446,081

(22) Filed: Jan. 12, 2026

(30) Foreign Application Priority Data
Jan. 14, 2025 (JP) 2025-004919
Nov. 6, 2025 (JP) 2025-187451
Dec. 25, 2025 (JP) 2025-281850

(51) Int. Cl. C12M 1/00; C12M 1/34

(52) U.S. Cl. CPC C12M 23/40; C12M 41/40; C12M 47/02

(57) ABSTRACT

A cell production device includes: a supply component that supplies a first liquid that includes a first cell; a separation component that separates the first cell from the first liquid supplied from the supply component; and an infection component that causes the first cell separated in the separation component to be infected with a virus to generate a second cell. The supply component, the separation component, and the infection component are connected by a flow path.

### CROSS REFERENCE TO RELATED APPLICATIONS

The present application is based on and claims priority of Japanese Patent Application No. 2025-004919 filed on Jan. 14, 2025, Japanese Patent Application No. 2025-187451 filed on Nov. 6, 2025, and Japanese Patent Application No. 2025-281850 filed on Dec. 25, 2025. The entire disclosures of the above-identified applications, including the specification, drawings and claims are incorporated herein by reference in their entirety.

### FIELD

The present disclosure relates to a cell production device, a vessel disposed in the cell production device, and a single-use kit.

### BACKGROUND

Stem cells such as induced pluripotent stem cells (iPS cells) and embryonic stem cells (ES cells) are known as pluripotent cells that can be produced from the cells of tissues included in, e.g., human skin, organs, and blood. In particular, iPS cells can be produced using cells derived from the patient to be treated, and then differentiated into the cells of each tissue. Thus, in regenerative medicine, there are expectations for iPS cells to be used as transplant materials in autologous transplants, for which rejection is infrequent.

For example, when producing iPS cells from blood, hematopoietic stem cells are extracted from the blood, and the extracted hematopoietic stem cells are infected with a virus by using a viral vector. This makes it possible to produce iPS cells by introducing iPS genes into hematopoietic stem cells. Furthermore, when iPS cells obtained in this way are to be used as transplant materials or the like, the iPS cells are propagated through culturing. Moreover, by inducing differentiation of the propagated iPS cells into T cells, for example, the T cells can be used as, e.g., immune cells such as individualized anti-cancer T cells.

When iPS cells are generated from blood, first it is necessary to separate and extract hematopoietic stem cells from the blood, as described above. In this case, a technique of separating hematopoietic stem cells from blood by means of magnetic force using magnetic beads or the like is known. For example, Patent Literature (PTL) 1 discloses a method in which magnetized cells are separated from a cell suspension, e.g., blood.

Using a single device to automatically generate iPS cells from blood that includes hematopoietic stem cells, and to automatically culture and propagate those iPS cells, is being investigated. In this case, for example, iPS cells are generated by: in a single device, supplying a material and a reagent to a vessel and separating hematopoietic stem cells from blood; infecting the separated hematopoietic stem cells with a virus to reprogram cells; and culturing the cells by using a culture medium.

### CITATION LIST

### Patent Literature

PTL 1: Japanese Unexamined Patent Application Publication (Translation of PCT Application) No. 2013-517763

### SUMMARY

### Technical Problem

However, using conventional devices, producing cells such as iPS cells without exposing these cells to contaminants is difficult.

The present disclosure was made in view of such problems, and provides a cell production device and the like that can establish cells such as iPS cells without exposing these cells to contaminants.

### Solution to Problem

One aspect of a cell production device according to the present disclosure includes: a supply component that supplies a first liquid that includes a first cell; a separation component that separates the first cell from the first liquid supplied from the supply component; and an infection component that causes the first cell separated in the separation component to be infected with a virus to generate a second cell, wherein the supply component, the separation component, and the infection component are connected, by a flow path.

Furthermore, one aspect of a vessel according to the present disclosure is a vessel disposed in the cell production device described above.

Furthermore, one aspect of a single-use kit according to the present disclosure is a single-use kit that is replaceable and is used in a cell production device that produces a cell such as an iPS cell, the single-use kit comprising: a liquid-holding vessel from which a first liquid that includes a first cell is supplied; a separation vessel in which the first cell is separated from the first liquid supplied from the liquid-holding vessel; and an infection vessel in which the first cell separated in the separation vessel is caused to be infected with a virus to generate a second cell, wherein the liquid-holding vessel, the separation vessel, and the infection vessel are connected, by a flow path.

### Advantageous Effects

The present disclosure makes it possible to produce cells such as iPS cells without exposing these cells to contaminants.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a diagram illustrating the configuration of a cell production device according to Embodiment 1.

FIG. 2 is a diagram illustrating the routes of a flow path in the cell production device according to Embodiment 1.

FIG. 3 is a diagram illustrating a single-use kit of the cell production device according to Embodiment 1.

FIG. 4 is a diagram schematically illustrating steps in a cell production method according to Embodiment 1.

FIG. 5 is a diagram illustrating details of the steps in the cell production method according to Embodiment 1.

FIG. 6A is a diagram that illustrates the dependence of the pressure during feeding and the pressure after feeding completion on the flow path length, when the liquid feeding flow rate is 0.2 ml/s.

FIG. 6B is a diagram that illustrates the dependence of the pressure during feeding and the pressure after feeding completion on the flow path length, when the liquid feeding flow rate is 0.3 ml/s.

FIG. 6C is a diagram that illustrates the dependence of the pressure during feeding and the pressure after feeding completion on the flow path length, when the liquid feeding flow rate is 0.4 ml/s.

FIG. 7 is a diagram that illustrates the configuration of a cell production device according to Embodiment 2.

### DESCRIPTION OF EMBODIMENTS

Hereinafter, specific exemplary embodiments of the present disclosure are described with reference to the accompanying Drawings. It should be noted that each of the exemplary embodiments described below shows a specific example. Thus, the numerical values, shapes, materials, constituent elements, the arrangement and connection of the constituent elements, steps, the processing order of the steps, etc. shown in the following exemplary embodiments are mere examples, and therefore do not limit the scope of the present disclosure. Therefore, among the constituent elements in the following exemplary embodiments, those not recited in any one of the independent claims are described as optional elements.

Furthermore, the respective figures are schematic diagrams and are not necessarily precise illustrations. Furthermore, in the figures, elements that are substantially the same are given the same reference signs, and overlapping descriptions are omitted or simplified.

### Embodiment 1

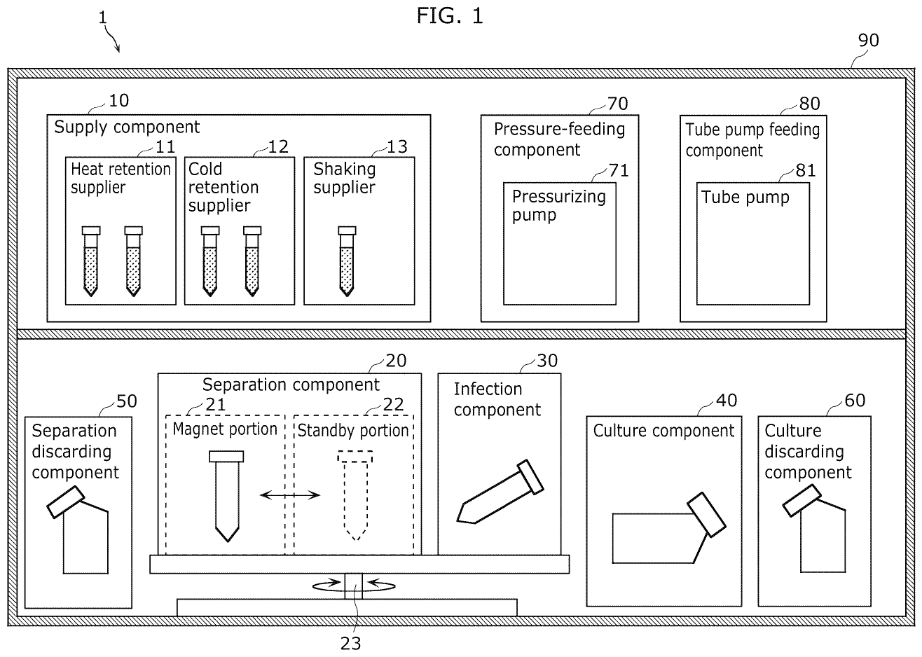

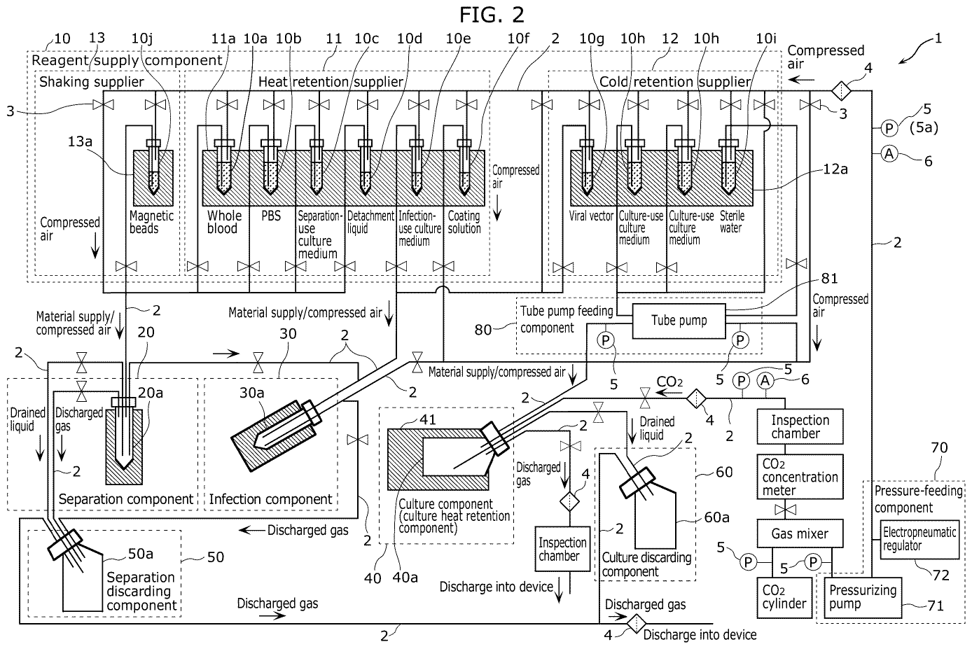

First, the configuration of cell production device 1 according to Embodiment 1 of the present disclosure is described with reference to FIG. 1 and FIG. 2. FIG. 1 is a diagram illustrating the configuration of cell production device 1 according to Embodiment 1. FIG. 2 is a diagram illustrating the routes (tube arrangement routes) of a flow path in cell production device 1 according to Embodiment 1.

Cell production device 1 is a device capable of producing cells that serve as targets (target cells). In the present embodiment, the target cells are iPS cells. Thus, cell production device 1 produces iPS cells. The iPS cells are produced from hematopoietic stem cells included in blood. Specifically, the iPS cells can be produced by causing hematopoietic stem cells extracted from blood to be infected with a virus by using a viral vector, and introducing iPS genes into the hematopoietic stem cells. Therefore, cell production device 1 includes: a mechanism for extracting and separating, from blood, hematopoietic stem cells that serve as the basis for iPS cells; a mechanism for imparting iPS genes to the extracted hematopoietic stem cells; and a mechanism for culturing the cells to which the iPS genes have been imparted. Cell production device 1 is capable of automatically and continuously performing a series of steps until producing iPS cells from blood.

As illustrated in FIG. 1, cell production device 1 includes: supply component 10 that supplies various reagents, materials, and a liquid that includes the cells serving as the basis for the target cells; separation component 20 that extracts and separates the cells from the liquid that includes the cells and was supplied from supply component 10; infection component 30 that causes the cells separated in separation component 20 to be infected to generate cells for which reprogramming has started; and culture component 40 that generates the target cells by culturing the cells for which reprogramming has started. The cells for which reprogramming has started are described in detail later.

In addition, cell production device 1 includes separation discarding component 50, culture discarding component 60, pressure-feeding component 70, and tube pump feeding component 80.

As illustrated in FIG. 1, supply component 10, separation component 20, infection component 30, culture component 40, separation discarding component 50, culture discarding component 60, pressure-feeding component 70, and tube pump feeding component 80 are disposed on support frame 90. Support frame 90 is, for example, a metal frame.

Supply component 10, pressure-feeding component 70, and tube pump feeding component 80 are disposed in an upper level of support frame 90. On the other hand, separation component 20, infection component 30, culture component 40, separation discarding component 50, and culture discarding component 60 are disposed in a lower level of support frame 90. Thus, supply component 10, pressure-feeding component 70, and tube pump feeding component 80 are located higher than separation component 20, infection component 30, culture component 40, separation discarding component 50, and culture discarding component 60. Thus disposing components such as supply component 10, which serve as feeding origins for liquid, vertically higher than components such as separation component 20 and infection component 30, which serve as feeding destinations for liquid, makes it possible to establish a height difference between the feeding origins and the feeding destinations. This makes it possible to facilitate the feeding of liquid in accordance with Bernoulli's principle, as well as to mitigate pressure drops of the liquid in flow path 2.

Predetermined vessels are disposed in supply component 10, separation component 20, infection component 30, culture component 40, separation discarding component 50, and culture discarding component 60.

A plurality of liquid-holding vessels that are each a vessel holding a predetermined amount of a predetermined liquid beforehand are disposed in supply component 10. Supply component 10 supplies the liquid held in the liquid-holding vessels disposed in supply component 10 to separation component 20, infection component 30, culture component 40, and the like. In other words, supply component 10 is a feeding origin for liquid. Furthermore, in the present embodiment, when producing iPS cells, liquid such as the materials, the reagents, and the like that are held in the liquid-holding vessels is present in a very small amount of 50 ml or less. In other words, the materials, reagents, and the like used in producing iPS cells are present in a very small amount.

Supply component 10 has the function of supplying, to separation component 20, a first liquid that includes the first cells that serve as the basis for the target cells. In other words, supply component 10 serves as a cell supply component. In the present embodiment, supply component 10 supplies, to separation component 20, blood that is the first liquid including hematopoietic stem cells as first cells. In this case, as illustrated in FIG. 2, cell vessel 10a holding whole blood (the first liquid) that is a cell suspension including hematopoietic stem cells (the first cells) is disposed, in supply component 10, as a liquid-holding vessel holding a predetermined liquid. In other words, cell vessel 10a holds blood as the first liquid, and the first liquid that is blood includes at least hematopoietic stem cells as the first cells. Cell vessel 10a holds, for example, 10 ml of whole blood. In the present embodiment, it is thus possible to produce iPS cells from a very small amount of whole blood. It should be noted that in the present embodiment, the first liquid was blood, but as long as at least hematopoietic stem cells are included, the first liquid may be another liquid. For example, the first liquid may be peripheral blood mononuclear cells (PBMC) obtained by removing plasma components, red blood cells, platelets and granulocytes from blood.

Furthermore, supply component 10 also has the function of supplying various reagents, materials, and the like. In other words, supply component 10 also serves as a reagent supply component. Specifically, as illustrated in FIG. 2, PBS vessel 10b, separation-use culture medium vessel 10c, detachment liquid vessel 10d, infection-use culture medium vessel 10e, coating solution vessel 10 f, viral vector vessel 10g, culture-use culture medium vessel 10h, sterile water vessel 10i, and magnetic bead vessel 10j are disposed in supply component 10 as liquid-holding vessels in which reagents, materials, and the like are held as the predetermined liquids.

PBS vessel 10b is a vessel holding phosphate buffered saline (PBS). PBS is a buffer solution and is used when washing cells, diluting liquids, and the like. In the present embodiment, the PBS held in PBS vessel 10b is supplied to separation vessel 20a disposed in separation component 20. PBS vessel 10b holds, for example, 20 ml of PBS.

Separation-use culture medium vessel 10c is a vessel holding a culture medium to be used when separating hematopoietic stem cells from blood. Separation-use culture medium vessel 10c is a first culture medium vessel holding the culture medium to be supplied to separation vessel 20a disposed in separation component 20. Thus, when separating hematopoietic stem cells from blood using separation component 20, the culture medium held in separation-use culture medium vessel 10c is fed to separation vessel 20a disposed in separation component 20. Separation-use culture medium vessel 10 c holds, for example, 5 ml of culture medium.

Detachment liquid vessel 10d is a vessel holding detachment liquid (bead release liquid) that acts on cells having magnetic beads bound thereto, to detach the cells and the magnetic beads from each other. When detaching the cells and the magnetic beads from each other, the detachment liquid held in detachment liquid vessel 10d is supplied to separation vessel 20a disposed in separation component 20. Detachment liquid vessel 10d holds, for example, 1 ml of the detachment liquid.

Infection-use culture medium vessel 10e is a vessel holding a culture medium to be used when causing cells to be infected with a virus. Infection-use culture medium vessel 10e is a second culture medium vessel holding the culture medium to be supplied to infection vessel 30a disposed in infection component 30. Thus, when causing the cells to be infected with the virus, the culture medium held in infection-use culture medium vessel 10e is fed to infection vessel 30a disposed in infection component 30. Infection-use culture medium vessel 10e holds, for example, 5 ml of culture medium.

Coating solution vessel 10f is a vessel holding coating solution. Before culturing the cells, the coating solution held in coating solution vessel 10f is supplied to culture vessel 40a disposed in culture component 40. Coating solution vessel 10f holds, for example, 5 ml of coating solution.

Viral vector vessel 10g is a vessel holding a liquid including a viral vector for causing cells to be infected with a virus. The viral vector is an agent used in cell production. When causing the cells to be infected with the virus, the liquid including the viral vector held in viral vector vessel 10g is supplied to infection vessel 30a disposed in infection component 30. The viral vector is a vector including a virus that is used for imparting specific genes to cells. In the present embodiment, since the target cells are iPS cells, the viral vector is used for imparting iPS genes to hematopoietic stem cells included in blood. Viral vector vessel 10g holds, for example, 0.2 ml of the liquid including the viral vector.

Culture-use culture medium vessel 10h is a vessel holding a culture medium for culturing cells. Culture-use culture medium vessel 10h is a third culture medium vessel holding the culture medium to be supplied to culture vessel 40a disposed in culture component 40. Thus, when culturing the cells that have been infected in infection component 30 and for which reprogramming has started, the culture medium held in culture-use culture medium vessel 10h is fed to culture vessel 40a disposed in culture component 40. The culture medium is a culture solution that includes, e.g., nutrients necessary for cell growth. The culture medium may be either a natural culture medium or a synthetic culture medium. It should be noted that in the present embodiment, two culture-use culture medium vessels 10h are provided, and when culturing cells, the second culture-use culture medium vessel 10h is used when the first culture-use culture medium vessel 10h becomes empty. Each of the two culture-use culture medium vessels 10h holds, for example, 40 ml of the culture medium.

Sterile water vessel 10i is a vessel holding sterile water. Sterile water is water that has been sterilized. Before culturing the cells for which reprogramming has started, the sterile water held in sterile water vessel 10i is supplied to culture vessel 40a disposed in culture component 40. Sterile water vessel 10i holds, for example, 40 ml of sterile water.

Magnetic bead vessel 10j is a vessel holding liquid including a plurality of magnetic beads (beads having magnetic properties). The liquid including the magnetic beads that is held in magnetic bead vessel 10j is supplied to separation vessel 20a disposed in separation component 20. The magnetic beads are an example of magnetic particles for separating specific cells from a cell suspension. Specifically, the magnetic beads are adsorbed to and bind to specific cells included in the cell suspension. In the present embodiment, the magnetic beads have the function of binding to hematopoietic stem cells included in blood. The magnetic beads are magnetic particles for separating hematopoietic stem cells from blood. Magnetic bead vessel 10j holds, for example, 1 ml of the liquid including the magnetic beads. It should be noted that the diameter of one magnetic bead is, as an example, 4.5 μm.

These liquid-holding vessels disposed in supply component 10 are all closed vessels. As an example, the liquid-holding vessels disposed in supply component 10 are rigid vessels made of a light-transmissive resin material. The liquid-holding vessels each have a plurality of ports for supplying liquid or gas that is inside the liquid-holding vessel, discharging liquid or gas that is inside the liquid-holding vessel, and the like. In the present embodiment, the liquid-holding vessels each have two ports. One of the two ports in each liquid-holding vessel may be used as an air hole. A port that serves as an air hole thus becomes a pressure-release hole through which gas escapes. Thus, using a pump or the like to apply pressure or to suction via one of the two ports makes it possible to discharge, from the other of the two ports, a liquid held in a liquid-holding vessel. For example, using pressure-feeding component 70 to send, from one of the two ports, compressed air to a liquid-holding vessel makes it possible to discharge the liquid in the liquid-holding vessel from the liquid-holding vessel, from the other of the two ports.

It should be noted that the shapes and the materials of the liquid-holding vessels disposed in supply component 10 are not particularly limited. For example, the liquid-holding vessels disposed in supply component 10 may each be a spitz tube having a tapered bottom, may each be a vessel constituted from a resin material other than a light-transmissive resin material, or may each be constituted from a material other than a resin material. For example, the liquid-holding vessels may each be a vessel made of glass or stainless steel. Furthermore, the liquid-holding vessels may be not a rigid vessel, but a vessel having flexibility, such as a light-transmissive bag or the like.

In the present embodiment, each liquid-holding vessel holding a predetermined liquid is disposed in a vessel-holding device that is disposed in supply component 10. The detailed configuration of the vessel-holding device is described later. Each of the plurality of liquid-holding vessels is replaceable, and can be received in the vessel-holding device and removed from the vessel-holding device. For example, the plurality of liquid-holding vessels are replaced each time the target cells are produced.

It should be noted that the plurality of liquid-holding vessels may be disposed on each of a plurality of vessel attachment portions provided to supply component 10. In this case, for example, each of the vessel attachment portions has a structure that allows a vessel to be hung and held, and is provided to a predetermined location of support frame 90. Each of the plurality of liquid-holding vessels can be replaced by attaching the liquid-holding vessel to the vessel attachment portion, removing the vessel from the vessel attachment portion, and so forth.

Furthermore, supply component 10 includes: heat retention supplier 11 (a heat retention block) that includes heat retention device 11a; cold retention supplier 12 (a cold retention block) that includes cold retention device 12a; and shaking supplier 13 that includes shaking device 13a.

Heat retention supplier 11 is heated by heat retention device 11a and kept at a constant high temperature. As an example, heat retention supplier 11 is kept at a constant temperature within the range of 35° C. to 40° C. Cell vessel 10a, PBS vessel 10b, separation-use culture medium vessel 10c, detachment liquid vessel 10d, infection-use culture medium vessel 10e, and coating solution vessel 10f are disposed in heat retention supplier 11. Thus, cell vessel 10a, PBS vessel 10b, separation-use culture medium vessel 10c (the first culture medium vessel), detachment liquid vessel 10d, infection-use culture medium vessel 10e (the second culture medium vessel), and coating solution vessel 10f are subjected to heat retention by heat retention device 11a so as to be at a constant high temperature. Heat retention device 11a may be configured as a vessel-holding device in which these liquid-holding vessels are disposed.

Cold retention supplier 12 is cooled by cold retention device 12a and kept at a low temperature. As an example, cold retention supplier 12 is kept at a constant temperature within the range of 2° C. to 6° C. Viral vector vessel 10g, culture-use culture medium vessel 10 h, and sterile water vessel 10i are disposed in cold retention supplier 12. Thus, viral vector vessel 10g, culture-use culture medium vessel 10h (the third culture medium vessel), and sterile water vessel 10i are subjected to cold retention by cold retention device 12a so as to be at a constant low temperature. In the present embodiment, cold retention device 12a is configured as vessel-holding device 100 in which these liquid-holding vessels are disposed.

Thus subjecting cell vessel 10a and magnetic bead vessel 10j to heat retention using heat retention device 11a and subjecting viral vector vessel 10g to cold retention using cold retention device 12a makes it possible to keep cell vessel 10a and magnetic bead vessel 10j at a constant high temperature and to keep viral vector vessel 10g at a constant low temperature, without being influenced by seasonal or daily temperature fluctuations or by temperature fluctuations due to the environment in which cell production device 1 is installed. This makes it possible to stabilize and unify the activity of the materials and the reagents. Furthermore, subjecting viral vector vessel 10g, culture-use culture medium vessel 10h, and sterile water vessel 10i to cold retention makes it possible to inhibit the occurrence and propagation of microbes.

Furthermore, in the present embodiment, separation-use culture medium vessel 10c and infection-use culture medium vessel 10e are subjected to heat retention by heat retention device 11a, and culture-use culture medium vessel 10h is subjected to cold retention by cold retention device 12a. Thus performing heat retention or cold retention according to the usage purpose, even for the same culture medium, makes it possible to supply, to a predetermined vessel, the culture medium at an appropriate temperature suited to that purpose. This makes it possible to efficiently produce iPS cells.

Shaking supplier 13 is shaken (oscillated) using shaking device 13a. Magnetic bead vessel 10j is disposed in shaking supplier 13. Thus, magnetic bead vessel 10j is shaken by shaking device 13a. Furthermore, shaking supplier 13 may be heated by a heat retention device and kept at a constant high temperature. For example, shaking supplier 13 may, similarly to heat retention supplier 11, be kept at a constant high temperature within the range of 35° C. to 40° C. Thus, magnetic bead vessel 10j is subjected to heat retention so as to be at a constant high temperature.

Separation component 20 has the function of separating hematopoietic stem cells (the first cells) from blood (the first liquid) supplied from supply component 10. In the present embodiment, the hematopoietic stem cells are extracted and separated from the blood using magnetic beads.

Specifically, first, separation vessel 20a is disposed, as an empty vessel, in separation component 20. Blood from cell vessel 10a disposed in supply component 10 is fed to separation vessel 20a, and a liquid that includes magnetic beads and is from magnetic bead vessel 10j disposed in supply component 10 is fed to separation vessel 20a. In other words, separation vessel 20a holds the blood fed from cell vessel 10a, and also holds the liquid fed from magnetic bead vessel 10j. The magnetic beads thus bind to the hematopoietic stem cells included in the blood. Then, by bringing a magnet close to separation vessel 20a, the hematopoietic stem cells to which the magnetic beads are bound are attracted to the magnet, thus making it possible to separate the hematopoietic stem cells from the blood.

In the present embodiment, separation component 20 includes magnet portion 21 and standby portion 22. Magnet portion 21 includes: a first housing that has a concave portion that receives separation vessel 20a; and a magnet provided to the first housing. The magnet in magnet portion 21 is, for example, a permanent magnet, and is disposed to surround separation vessel 20a disposed in the first housing. Standby portion 22 includes a second housing that has a concave portion that receives separation vessel 20a. Separation vessel 20a before being transferred to magnet portion 21 is disposed in standby portion 22. In other words, in standby portion 22, blood from cell vessel 10a is fed to separation vessel 20a, and the liquid that includes the magnetic beads and is from magnetic bead vessel 10j is fed to separation vessel 20a, whereupon the magnetic beads bind to the hematopoietic stem cells. Subsequently, separation vessel 20a in standby portion 22 is transferred to magnet portion 21. This makes it possible to separate, from the blood, the hematopoietic stem cells to which the magnetic beads are bound, by using the magnetic force of the magnet in magnet portion 21.

Separation component 20 is configured to be turnable. Specifically, as illustrated in FIG. 1, cell production device 1 has turning mechanism 23 for causing separation component 20 to turn. It should be noted that as used herein, turning may refer to turning about an internal axis (rotation) and/or turning about an external axis (revolution).

Infection component 30 has the function of causing the hematopoietic stem cells (first cells) separated by separation component 20 to be infected with the virus, to generate cells for which reprogramming has started (the second cells). In the present embodiment, to generate the cells for which reprogramming has started, iPS genes are introduced into hematopoietic stem cells extracted from blood by causing the hematopoietic stem cells to be infected with the virus by using a viral vector.

Specifically, first, infection vessel 30a is disposed, as an empty vessel, in infection component 30. A liquid that includes the hematopoietic stem cells and is from separation vessel 20a disposed in separation component 20 is fed to infection vessel 30a, and a liquid that includes a viral vector and is from viral vector vessel 10g disposed in supply component 10 is fed to infection vessel 30a. In other words, infection vessel 30a holds the liquid that includes the hematopoietic stem cells and is fed from separation vessel 20a, and also holds the liquid that includes the viral vector and is fed from viral vector vessel 10g. In infection vessel 30a, the hematopoietic stem cells are thus infected with the virus and the hematopoietic stem cell reprogramming is started. In other words, the cells for which reprogramming has started are generated. It is to be noted that in the present embodiment, in infection component 30, the cells for which reprogramming has started may be iPS cells, and the iPS cells generated in infection component 30 may be cultured in culture component 40.

Infection component 30 is configured to be turnable. In the present embodiment, infection component 30 turns by using turning mechanism 23 for causing separation component 20 to turn. In other words, the turning mechanism for causing infection component 30 to turn and the turning mechanism for causing separation component 20 to turn are shared. Thus, separation component 20 and infection component 30 turn simultaneously. It should be noted that the turning mechanism for causing infection component 30 to turn and the turning mechanism for causing separation component 20 to turn may be separate turning mechanisms.

Culture component 40 has the function of culturing the cells for which reprogramming has started, generated in infection component 30. In the present embodiment, culture component 40 cultures cells for which reprogramming has started that have been generated in infection component 30, and iPS cells are produced.

Specifically, first, culture vessel 40a is disposed, as an empty vessel, in culture component 40. Liquid that includes the cells for which reprogramming has started and is from infection vessel 30a disposed in infection component 30 is fed to culture vessel 40a, and culture medium from culture-use culture medium vessel 10h disposed in supply component 10 is fed to culture vessel 40a. In other words, culture vessel 40a holds the liquid that includes the cells for which reprogramming has started and is fed from infection vessel 30a, and also holds the culture medium fed from culture-use culture medium vessel 10h.

Furthermore, culture component 40 includes heat retention device 41. This makes it possible to heat culture vessel 40a disposed in culture component 40 by using heat retention device 41, to keep culture vessel 40a at a constant high temperature. For example, culture vessel 40a is kept at a constant high temperature within the range of 35° C. to 40° C.

Separation discarding component 50 has the function of collecting liquid that has become unneeded in separation component 20. Discard vessel 50a that is an empty vessel is disposed in separation discarding component 50. Discard vessel 50a is a liquid collection vessel for collecting liquid that has become unneeded in separation component 20. Specifically, discard vessel 50a collects liquid discharged from separation vessel 20a disposed in separation component 20.

Culture discarding component 60 has the function of collecting liquid that has become unneeded in culture component 40. Discard vessel 60a that is an empty vessel is disposed in culture discarding component 60. Discard vessel 60a is a liquid collection vessel for collecting liquid that has become unneeded in culture component 40. Specifically, discard vessel 60a collects liquid discharged from culture vessel 40a disposed in culture component 40.

When iPS cells are produced, empty vessels are thus disposed in separation component 20, infection component 30, culture component 40, separation discarding component 50, and culture discarding component 60. Separation vessel 20a disposed in separation component 20, infection vessel 30a disposed in infection component 30, and culture vessel 40a disposed in culture component 40 are vessels for processing cells, and for example, are each a light-transmissive vessel, such as a spitz tube or a flask, made of a light-transmissive resin material. As an example, separation vessel 20a disposed in separation component 20 is a spitz tube, and infection vessel 30a disposed in infection component 30 and culture vessel 40a disposed in culture component 40 are T-flasks. Furthermore, discard vessel 50a disposed in separation discarding component 50 and discard vessel 60a disposed in culture discarding component 60 are each, for example, a light-transmissive vessel, such as a flask, made of a light-transmissive resin material.

Separation vessel 20a, infection vessel 30a, culture vessel 40a, discard vessel 50a, and discard vessel 60a are all closed vessels. As an example, separation vessel 20a, infection vessel 30a, culture vessel 40a, discard vessel 50a, and discard vessel 60a are each a rigid vessel made of a light-transmissive resin material. Each of separation vessel 20a, infection vessel 30a, culture vessel 40a, discard vessel 50a, and discard vessel 60a has at least two ports for supplying liquid or gas into the vessel or discharging liquid or gas that is inside the vessel. In these vessels, one of the at least two ports can be used as an air hole. This results in the port that serves as an air hole becoming a pressure-release hole through which gas escapes. Thus, using a pump or the like to apply pressure or to suction via one of the at least two ports makes it possible to supply liquid into the vessel or discharge liquid that is inside the vessel, from another of the at least two ports. For example, liquid can be supplied into the vessel and liquid inside the vessel can be discharged, by sending compressed air to the vessel using pressure-feeding component 70.

It should be noted that the shape and materials of each of separation vessel 20a, infection vessel 30a, culture vessel 40a, discard vessel 50a, and discard vessel 60a are not particularly limited. For example, separation vessel 20a, infection vessel 30a, culture vessel 40a, discard vessel 50a, and discard vessel 60a may each be a vessel made of glass or stainless steel, or may each be not a rigid vessel, but a vessel having flexibility, such as a light-transmissive bag or the like.

Furthermore, cell production device 1 has a plurality of vessel attachment portions for attaching these vessels. For example, each of the plurality of vessel attachment portions has a structure that allows a vessel to be hung and held, and is provided to a predetermined location of support frame 90. These vessels are replaceable, and can be attached to the vessel replacement portions or removed from the vessel replacement portions. For example, these vessels are replaced each time the iPS cells are produced.

Pressure-feeding component 70 has the function of feeding liquid by pressurization (in other words, by applying pressure). As an example, pressure-feeding component 70 includes pressurizing pump 71. In the present embodiment, pressurizing pump 71 is a diaphragm pump. For example, pressure-feeding component 70 feeds liquid from a feeding origin to a feeding destination by supplying compressed air into flow path 2 to perform pressurization. Specifically, pressure-feeding component 70 feeds liquid from supply component 10 to separation component 20, infection component 30, and culture component 40 by supplying compressed air using pressurization. Furthermore, by supplying compressed air using pressurization, pressure-feeding component 70 feeds liquid from separation component 20 to infection component 30, feeds liquid from infection component 30 to culture component 40, feeds liquid from separation component 20 to separation discarding component 50, and feeds liquid from culture component 40 to culture discarding component 60. It should be noted that pressure-feeding component 70 includes electropneumatic regulator 72 that adjusts the pressure of the liquid that flows through flow path 2.

Liquids such as the materials, reagents, and the like used in producing iPS cells are present in a very small amount, but it is difficult to feed the very small amount of liquid in its entirety using only a tube pump. Accordingly, as in the present embodiment, feeding the liquid using pressurization instead of suction makes it possible to easily feed the liquid from supply component 10 to separation component 20 and infection component 30, even if the liquid is present in a very small amount. In other words, feeding the liquid by using pressurization makes it possible to transfer all of the liquid in the feeding origin vessels to the feeding destination vessels. This makes it possible to feed the liquid without leaving any liquid in the vessel that serves as the feeding origin, even when the liquid to be fed is very small in amount. Using air that has been filtered to the highest level of cleanliness (for example, a sterile state) makes it possible to inhibit contamination. On the other hand, when feeding liquid by using suction, even in a closed vessel, there is a risk of contamination from the outside environment, which has the lowest level of cleanliness. Thus, it can be stated that pressurized feeding has a lower risk.

When the liquid is fed from the feeding origin to the feeding destination via flow path 2 by pressure-feeding component 70, in the present embodiment, the liquid is fed in a very small amount of 50 ml or less. Thus, the flow rate (the feeding speed) for feeding of the liquid is preferably 0.1 ml/s or greater and 0.4 ml/s or less.

Tube pump feeding component 80 has tube pump 81 that, using a roller, pushes out liquid that has been sucked into the tube. Tube pump 81 is able to slowly feed a small amount of liquid. Tube pump 81 feeds the liquid of the liquid-holding vessels disposed in cold retention supplier 12 of supply component 10 to infection vessel 30a of infection component 30, and culture vessel 40a of culture component 40. For example, tube pump 81 feeds the culture medium of culture-use culture medium vessel 10h and the sterile water of sterile water vessel 10i to culture vessel 40a.

Cell production device 1 is a closed device, and as illustrated in FIG. 2, supply component 10, separation component 20, and infection component 30 are connected by flow path 2 (a tube arrangement), such that cell production device 1 is continuously in a closed state. In other words, the vessels disposed in supply component 10, separation component 20, and infection component 30, respectively, are linked by flow path 2. In FIG. 2, flow path 2 is illustrated by a thick solid line.

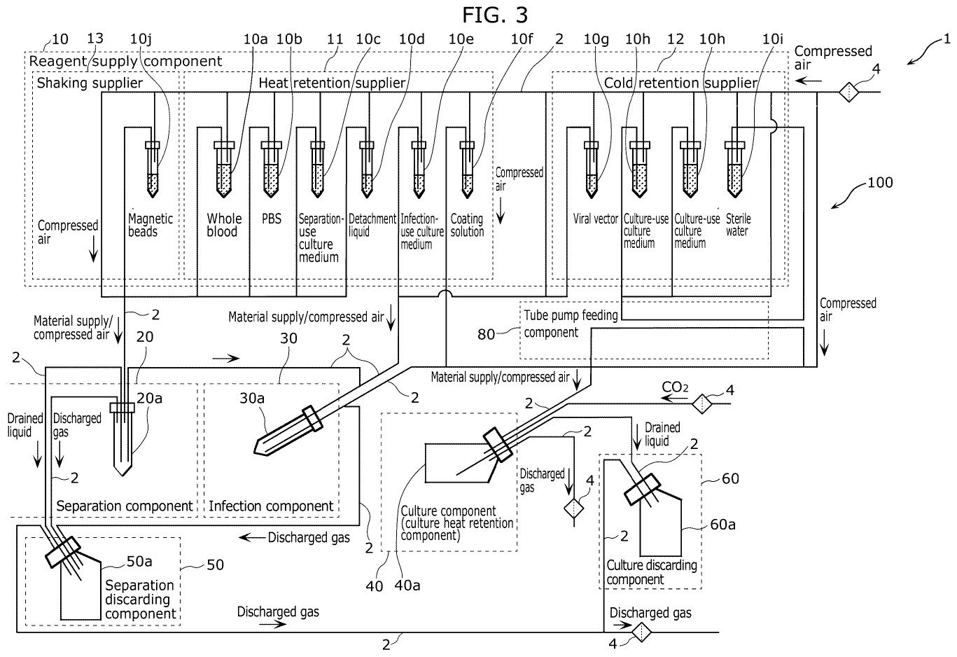

As illustrated in FIG. 3, these vessels and flow path 2 that links these vessels are single-use kit 100 that is replaced each time the iPS cells are produced. In other words, cell production device 1 is a device that produces cells by single-use kit 100 being disposed therein, each time. FIG. 3 is a diagram illustrating single-use kit 100 of cell production device 1 according to Embodiment 1.

Thus, when culturing cells, the part of cell production device 1 that becomes contaminated by cells or the like can be replaced each time as single-use kit 100, thereby facilitating the production of iPS cells. For example, when producing iPS cells from autologous hematopoietic stem cells using cell production device 1, after producing iPS cells once, the single-use kit must be replaced when subsequently producing iPS cells using another person's cells, in order to prevent the cells from mixing. Cell production device 1 according to Embodiment 1 enables the replacement of single-use kit 100, and thus makes it possible to easily produce personalized iPS cells.

Single-use kit 100 includes: the liquid-holding vessels disposed in supply component 10 (cell vessel 10a, PBS vessel 10b, separation-use culture medium vessel 10c, detachment liquid vessel 10d, infection-use culture medium vessel 10e, coating solution vessel 10f, viral vector vessel 10g, culture-use culture medium vessel 10h, sterile water vessel 10i, and magnetic bead vessel 10j); separation vessel 20a disposed in separation component 20; infection vessel 30a disposed in infection component 30; culture vessel 40a disposed in culture component 40; discard vessel 50a disposed in separation discarding component 50; discard vessel 60a disposed in culture discarding component 60; and flow path 2 that links these vessels. These vessels and flow path 2 are sterilized in advance.

Using single-use kit 100 in which the plurality of vessels that have been sterilized in advance are thus linked by flow path 2 makes it unnecessary to sterilize or wash the plurality of vessels or flow path 2 that links the plurality of vessels. Thus, the risk of the cells becoming contaminated is decreased, enabling cell production operations in which a sterile state is maintained. Furthermore, linking the plurality of vessels and flow path 2 in advance makes it simpler to dispose each component in cell production device 1, making it possible to shorten vessel installation times.

In single-use kit 100 of the present embodiment, flow path 2 connects, as a closed system, supply component 10, separation component 20, infection component 30, and culture component 40. In other words, flow path 2 links: the liquid-holding vessels disposed in supply component 10; separation vessel 20a disposed in separation component 20; infection vessel 30a disposed in infection component 30; and culture vessel 40a disposed in culture component 40.

Here, a closed system means that each element of cell production device 1 linked by flow path 2 is in a state blocked off from the external environment via filter 4, described later. In the present embodiment, the closed system indicates a state in which microbes, particles, and the like that are foreign substances in gases such as carbon dioxide or air are trapped upon passing through filter 4, whereby a sterile state can be secured. It should be noted that the closed system is not limited to filter 4 being used. For example, the closed system may be such that the configuration of vessels involves linkage by flow path 2 in a completely sealed state.

It should be noted that flow path 2 further connects, as a closed system, separation discarding component 50 and culture discarding component 60. In other words, flow path 2 further links discard vessel 50a disposed in separation discarding component 50 and discard vessel 60a disposed in culture discarding component 60.

The plurality of vessels connected via flow path 2 (the liquid-holding vessels, separation vessel 20a, infection vessel 30a, culture vessel 40a, discard vessel 50a, and discard vessel 60a) define a closed space, and more specifically define a space that in a sterile state due to being, e.g., closed. However, a slight margin of error that is unintended, such as a drop in the airtightness of the plurality of vessels, is included in the closed space in the present disclosure.

Flow path 2 is a tube arrangement through which fluids such as liquid and/or gas pass. Flow path 2 is a thin, rigid tube arrangement. Specifically, the inner diameter of flow path 2 is 0.5 mm or greater and 5 mm or less. Using, as flow path 2, a thin, rigid tube arrangement having an inner diameter of 0.5 mm or greater and 5 mm or less makes it possible to introduce a pressure drop that is sufficient to realize a slow flow rate. In the present embodiment, as thin, rigid flow path 2, a tube made of polyethylene (PE) and having an inner diameter of 1 mm was used. In this case, flow path 2 may be a tube made of polyethylene subjected to a water-repellent treatment. It should be noted that flow path 2 may be made not of polyethylene, but of Teflon (registered trademark) or the like. Teflon (registered trademark) has water-repellent properties. Thus, using Teflon (registered trademark) for flow path 2 makes it possible for flow path 2 to be a tube that has water-repellent properties. Furthermore, flow path 2 need not be a rigid tube arrangement. For example, flow path 2 may be a tube that has flexibility and is made of silicone or the like. Furthermore, flow path 2 is not limited to being made of resin, and may be made of metal. Moreover, flow path 2 is constituted from a plurality of tubes in order to link the plurality of vessels to each other.

A plurality of valves 3 are disposed in cell production device 1. The plurality of valves 3 are opening/closing valves that control the opening and closing of flow path 2. In the present embodiment, valves 3 are pinch valves. Controlling the plurality of valves 3 at predetermined timings makes it possible to pass through or stop liquid or gas at the locations at which valves 3 are disposed in flow path 2. When using pinch valves as valves 3, flow path 2 is flexible at least at portions that connect to valves 3. It is to be noted that in FIG. 2, the reference sign “3” is appended only to two valves of the plurality of valves 3. In other words, the valves having no reference symbol appended thereto similarly open and close flow path 2 at predetermined timings. It should be noted that in the present embodiment, valves 3 were disposed in cell production device 1, but this is not intended to be limiting. For example, valves 3 may be disposed in flow path 2. In this case, valves 3 may be air valves or solenoid valves.

Furthermore, a plurality of filters 4 are disposed in flow path 2. Each of the plurality of filters 4 is, for example, a filter that secures sufficient sterility and dust collection performance. As an example, each filter 4 is a hydrophobic membrane filter, a high efficiency particulate air (HEPA) filter, or the like. Each filter 4 traps foreign matter included in gas that passes through that filter 4. Each filter 4 is provided at a location through which gas passes inside flow path 2, provided at a location at which gas is discharged from flow path 2 to the outside, or the like. This makes it possible to keep the device interior of cell production device 1 and the surrounding environment outside of cell production device 1 in a sterile state suitable for use at a cell culture and processing facility.

Furthermore, a plurality of pressure gauges 5 are disposed in cell production device 1. Each of the plurality of pressure gauges 5 measures the pressure of the liquid that flows in flow path 2 at the location at which that pressure gauge 5 is disposed. Pressure gauge 5a of the plurality of pressure gauges 5 measures the pressure of the liquid fed by pressure-feeding component 70. Cell production device 1 includes a determiner that determines that the feeding of the liquid is completed, when the pressure measured by pressure gauge 5a decreases.

Furthermore, a plurality of flow rate meters 6 are disposed in cell production device 1. Each of the plurality of flow rate meters 6 measures the flow rate of the liquid that flows in flow path 2 at the location at which that flow rate meter 6 is disposed.

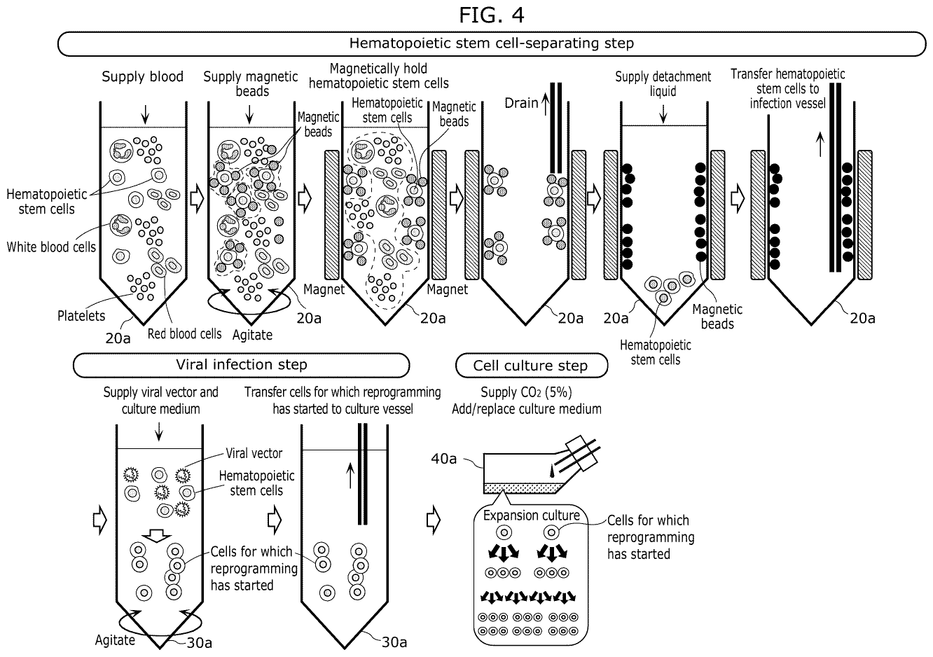

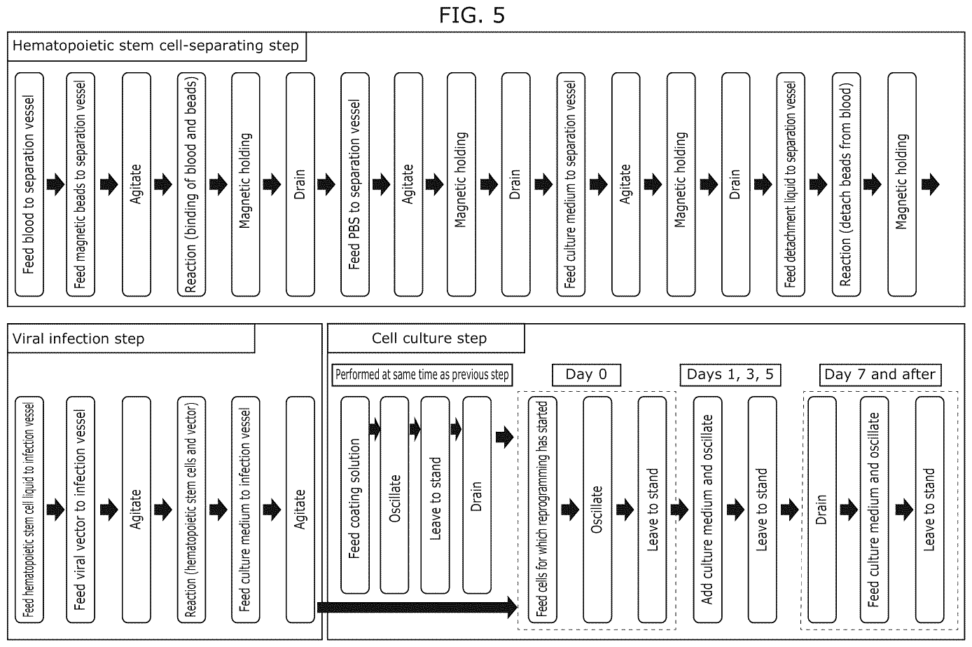

Next, an overview of a cell production method in which cell production device 1 illustrated in FIG. 1 and FIG. 2 is used is described with reference to FIG. 4 and FIG. 5, while referring to FIG. 2. In the present embodiment, using cell production device 1, iPS cells are produced. Thus, a cell production method in which cell production device 1 is used is described. FIG. 4 and FIG. 5 are diagrams for illustrating the cell production method in Embodiment 1. FIG. 4 schematically illustrates steps in the cell production method. Furthermore, FIG. 5 illustrates the details of each step.

It should be noted that in the steps below, the opened/closed states of the plurality of valves 3 are appropriately controlled such that liquid or gas flows only to a predetermined flow path 2 and is supplied to a predetermined supply destination. For example, when feeding liquid from a predetermined vessel to another vessel, valves other than the valves provided in flow path 2 between the feeding origin of the liquid (supply component 10, separation component 20, or infection component 30) and the feeding destination of the liquid (separation component 20, infection component 30) are closed. This makes it possible to prevent interference in other steps.

First, each type of valve is disposed in cell production device 1. Specifically, PBS vessel 10b, separation-use culture medium vessel 10c, detachment liquid vessel 10d, infection-use culture medium vessel 10e, coating solution vessel 10 f, viral vector vessel 10g, culture-use culture medium vessel 10h, sterile water vessel 10i, and magnetic bead vessel 10j are disposed in supply component 10 as liquid-holding vessels that hold liquids in predetermined amounts.

Furthermore, separation vessel 20a that is empty is disposed in standby portion 22 of separation component 20, infection vessel 30a that is empty is disposed in infection component 30, culture vessel 40a that is empty is disposed in culture component 40, discard vessel 50a that is empty is disposed in separation discarding component 50, and discard vessel 60a that is empty is disposed in culture discarding component 60.

Then, after each type of vessel is disposed, as illustrated in FIG. 4 and FIG. 5, in cell production device 1, a step of separating hematopoietic stem cells from blood (a hematopoietic stem cell-separating step) is performed.

In the hematopoietic stem cell-separating step, first, blood that is whole blood is supplied to separation vessel 20a. Specifically, the blood held in cell vessel 10a disposed in heat retention supplier 11 of supply component 10 is fed to separation vessel 20a that is empty and disposed in separation component 20, by supplying compressed air to flow path 2 using pressure-feeding component 70, illustrated in FIG. 2. The blood includes hematopoietic stem cells, white blood cells, red blood cells, and platelets.

Next, as illustrated in FIG. 4 and FIG. 5, magnetic beads are supplied to separation vessel 20a. Specifically, liquid that includes magnetic beads and is held in magnetic bead vessel 10j disposed in shaking supplier 13 of supply component 10 is fed to separation vessel 20a in which blood is already held, by supplying compressed air to flow path 2 using pressure-feeding component 70, illustrated in FIG. 2.

Subsequently, the blood and the magnetic beads that are held in separation vessel 20a are agitated. Specifically, the blood and the magnetic beads inside separation vessel 20a are agitated, by causing separation component 20 to turn using turning mechanism 23, illustrated in FIG. 1. This makes it possible to cause the magnetic beads to be adsorbed to the hematopoietic stem cells included in the blood and to cause the hematopoietic stem cells and the magnetic beads to bind together. In this case, to promote a reaction in which the hematopoietic stem cells and the magnetic beads bind together, the hematopoietic stem cells and the magnetic beads are preferably left to stand for a certain time period (for example, 30 minutes).

Next, separation vessel 20a holding the blood and the magnetic beads is transferred from standby portion 22 to magnet portion 21. This makes it possible to attract to magnet portion 21 and hold in place the hematopoietic stem cells to which the magnetic beads are bound, by using the magnetic force of the magnets of magnet portion 21, as illustrated in FIG. 4 and FIG. 5. Specifically, it is possible to cause the hematopoietic stem cells to which the magnetic beads are bound to be held against the inner surface of separation vessel 20a.

Next, as illustrated in FIG. 4 and FIG. 5, the liquid inside separation vessel 20a is drained. Specifically, the liquid inside separation vessel 20a is fed to discard vessel 50a disposed in separation discarding component 50, by supplying compressed air to flow path 2 using pressure-feeding component 70, illustrated in FIG. 2. At this time, the hematopoietic stem cells to which the magnetic beads are bound are held against the inner surface of separation vessel 20a using the magnetic force of magnet portion 21, and thus remain inside separation vessel 20a. The hematopoietic stem cells are thus separated from the blood and extracted.

It should be noted that as illustrated in FIG. 5, subsequently, separation vessel 20a in which the hematopoietic stem cells have been held in place may be transferred to standby portion 22, and to wash the hematopoietic stem cells, PBS of PBS vessel 10b disposed in heat retention supplier 11 of supply component 10 may be fed to separation vessel 20a and agitated. In this case, subsequently, separation vessel 20a is again transferred to magnet portion 21, the hematopoietic stem cells to which the magnetic beads are bound are held against the inner surface of separation vessel 20a using the magnetic force of the magnets of magnet portion 21, and the liquid (PBS) inside separation vessel 20a is drained. In other words, the liquid inside separation vessel 20a is fed to discard vessel 50a.

Moreover, as illustrated in FIG. 5, subsequently, separation vessel 20a in which the hematopoietic stem cells have been held in place may be further transferred to standby portion 22, and to stabilize the cells, the culture medium of separation-use culture medium vessel 10c disposed in heat retention supplier 11 of supply component 10 may be fed to separation vessel 20a and agitated. In this case, subsequently, separation vessel 20a is again transferred to magnet portion 21, the hematopoietic stem cells to which the magnetic beads are bound are held against the inner surface of separation vessel 20a using the magnetic force of the magnets of magnet portion 21, and the liquid (culture medium) inside separation vessel 20a is drained. In other words, the liquid inside separation vessel 20a is fed to discard vessel 50a.

Next, as illustrated in FIG. 4 and FIG. 5, detachment liquid is supplied to separation vessel 20a. Specifically, the detachment liquid held in detachment liquid vessel 10d disposed in heat retention supplier 11 of supply component 10 is fed to separation vessel 20a, by supplying compressed air to flow path 2 using pressure-feeding component 70, illustrated in FIG. 2. This makes it possible to detach the magnetic beads and the hematopoietic stem cells to which the magnetic beads have bound from each other. In this case, to promote a reaction of causing the hematopoietic stem cells and the magnetic beads to detach from each other, the hematopoietic stem cells and the magnetic beads are preferably left to stand for a certain time period (for example, 30 minutes).

It should be noted that the step of detaching the hematopoietic stem cells and the magnetic beads from each other may be performed in magnet portion 21 or may be performed in standby portion 22, but in the case of performing the detaching step in standby portion 22, as illustrated in FIG. 5, separation vessel 20a is transferred to magnet portion 21, and the magnetic beads separated from the hematopoietic stem cells are held against the inner surface of separation vessel 20a.

After performing the step of separating the hematopoietic stem cells from the blood (the hematopoietic stem cell-separating step), as illustrated in FIG. 4 and FIG. 5, a step of causing the hematopoietic stem cells to be infected with the virus by using a viral vector (a viral infection step) is performed.

In the viral infection step, first, a liquid that includes the hematopoietic stem cells separated in the hematopoietic stem cell-separating step is transferred to infection vessel 30a disposed in infection component 30. Specifically, the hematopoietic stem cells inside separation vessel 20a of separation component 20 are transferred to infection vessel 30a that is empty and disposed in infection component 30, by supplying compressed air to flow path 2 using pressure-feeding component 70, illustrated in FIG. 2.

Next, as illustrated in FIG. 4 and FIG. 5, a liquid that includes a viral vector is supplied to infection vessel 30a. Specifically, the liquid that includes the viral vector and is held in viral vector vessel 10g disposed in cold retention supplier 12 of supply component 10 is fed to infection vessel 30a in which the hematopoietic stem cells are held, by supplying compressed air to flow path 2 using pressure-feeding component 70, illustrated in FIG. 2.

Subsequently, a liquid that includes the hematopoietic stem cells and the viral vector and is held in infection vessel 30a is agitated. Specifically, the liquid that includes the hematopoietic stem cells and the viral vector and is inside infection vessel 30a is agitated by causing infection component 30 to turn by using turning mechanism 23, illustrated in FIG. 1. The hematopoietic stem cells inside infection vessel 30a are thus infected with the virus by using the viral vector, and iPS genes are introduced into the hematopoietic stem cells. The hematopoietic stem cells thus become cells for which reprogramming has started. In other words, iPS cells are produced. It is to be noted that subsequently, as illustrated in FIG. 5, to promote the reaction of the viral infection between the hematopoietic stem cells and the viral vector, the hematopoietic stem cells and the viral vector are preferably left to stand for a certain time period (for example, 120 minutes).

Next, culture medium is supplied to infection vessel 30a. Specifically, the culture medium inside infection-use culture medium vessel 10e disposed in heat retention supplier 11 of supply component 10 is fed to infection vessel 30a, by supplying compressed air to flow path 2 using pressure-feeding component 70, illustrated in FIG. 2.

Subsequently, liquid that includes the cells for which reprogramming has started and is inside infection vessel 30a is agitated. Specifically, the liquid that includes the cells for which reprogramming has started and is inside infection vessel 30a is agitated by causing infection component 30 to turn by using turning mechanism 23, illustrated in FIG. 1.

After performing the step of causing the hematopoietic stem cells to be infected with the virus using the viral vector (the viral infection step), as illustrated in FIG. 4 and FIG. 5, a step of culturing the cells for which reprogramming has started (a cell culture step) is performed.

Before performing the cell culture step, a pre-treatment of culture vessel 40a is performed in advance, at the same time as the viral infection step, which is the previous step.

Specifically, as illustrated in FIG. 5, a coating solution is supplied to culture vessel 40a that is empty. Specifically, the coating solution held in coating solution vessel 10f disposed in heat retention supplier 11 of supply component 10 is fed to culture vessel 40a that is empty and disposed in culture component 40, by supplying compressed air to flow path 2 using pressure-feeding component 70, illustrated in FIG. 2. Subsequently, culture vessel 40a holding the coating solution is oscillated, then culture vessel 40a is left to stand for a certain time period (for example, 60 minutes), and after that, the coating solution inside culture vessel 40a is drained to discard vessel 60a by supplying compressed air to flow path 2 using pressure-feeding component 70. The coating solution thus coats the inner surface of culture vessel 40a. The coating solution (a scaffolding agent) coating culture vessel 40a makes it possible to culture the cells for which reprogramming has started while these cells are adhered to the bottom of culture vessel 40a.

In the cell culture step, first, the cells for which reprogramming has started produced in the viral infection step are transferred to culture vessel 40a disposed in culture component 40. In the present embodiment, the cells for which reprogramming has started are transferred to culture vessel 40a that is empty and whose inner surface is coated with the coating solution. Specifically, a liquid that includes the cells for which reprogramming has started and the culture medium and is inside infection vessel 30a of infection component 30 is fed to culture vessel 40a that is empty and disposed in culture component 40, by supplying compressed air to flow path 2 using pressure-feeding component 70, illustrated in FIG. 2.

Next, as illustrated in FIG. 5, culture vessel 40a holding the liquid that includes the cells for which reprogramming has started and the culture medium is oscillated, and subsequently, culture vessel 40a is left to stand for one day.

After this, on the first day, as illustrated in FIG. 5, culture medium is added to culture vessel 40a, and culture vessel 40a is oscillated and left to stand for two days. When adding culture medium to culture vessel 40a, by supplying compressed air to flow path 2 by using pressure-feeding component 70 illustrated in FIG. 2, culture medium held in culture-use culture medium vessel 10h disposed in cold retention supplier 12 of supply component 10 is fed to culture vessel 40a. It should be noted that instead of adding culture medium to culture vessel 40a, the culture medium inside culture vessel 40a may be drained once, and then new culture medium may be fed from culture-use culture medium vessel 10h to culture vessel 40a. In other words, the culture medium may be replaced.

On the third day, similarly, culture medium is added to culture vessel 40a, and culture vessel 40a is oscillated and left to stand for two days. On the fifth day, similarly, culture medium is added to culture vessel 40a or replaced, and culture vessel 40a is oscillated and left to stand for two days.

On the seventh day and afterward, as illustrated in FIG. 5, the culture medium inside culture vessel 40a is drained, culture medium is fed to culture vessel 40a, and culture vessel 40a is oscillated and left to stand for two days.

As described above, iPS cells can be produced by expansion culture of the cells for which reprogramming has started over several days to several weeks. Subsequently, while not illustrated, the produced iPS cells may be collected from culture vessel 40a to a collection vessel.

Furthermore, during the period of the cell culture step described above, CO₂ gas including carbon dioxide is continuously supplied to culture vessel 40a, to keep the carbon dioxide concentration inside culture vessel 40a constant. For example, the concentration of the carbon dioxide included in the gas inside culture vessel 40 a is preferably kept at 2% to 10% (preferably 5%). In this case, when carbon dioxide is continuously supplied, the culture medium inside culture vessel 40a dries out and gradually decreases, and the concentration of the culture medium changes. Accordingly, to keep the concentration of the culture medium inside culture vessel 40a at or above a certain level, culture medium from culture-use culture medium vessel 10h and/or sterile water from sterile water vessel 10i may be fed, in increments of small amounts, to culture vessel 40a using tube pump 81 of tube pump feeding component 80. Since the concentration of the culture medium inside culture vessel 40a can be kept at or above a predetermined level and the amount of moisture in the culture medium of culture vessel 40a can be kept at or above a certain level, cells can be efficiently cultured. Furthermore, in the cell culture step, the culture medium of culture vessel 40a is preferably kept at a constant temperature (for example, 37° C.) using heat retention device 41. This makes it possible to efficiently produce iPS cells.

As described above, cell production device 1 according to the present embodiment includes: supply component 10 that supplies blood (the first liquid) that includes hematopoietic stem cells (the first cells); separation component 20 that separates the hematopoietic stem cells from the blood supplied from supply component 10; and infection component 30 that causes the hematopoietic stem cells separated in separation component 20 to be infected with a virus to generate cells for which reprogramming has started (the second cells). Supply component 10, separation component 20, and infection component 30 are connected by flow path 2.

Since supply component 10, separation component 20, and infection component 30 are thus connected, by flow path 2, as a closed system, iPS cells can be produced without being exposed to contaminants.

Furthermore, cell production device 1 according to the present embodiment further includes culture component 40 that cultures the cells for which reprogramming has started (the second cells) generated in infection component 30. Flow path 2 connects supply component 10, separation component 20, infection component 30, and culture component 40.

Since not only supply component 10, separation component 20, and infection component 30, but also culture component 40 is connected in a closed system by flow path 2, this configuration makes it possible for iPS cells to be produced without being exposed to contaminants.

Furthermore, in cell production device 1 according to the present embodiment, cell vessel 10a (a first vessel), magnetic bead vessel 10j (a second vessel), and viral vector vessel 10g (a third vessel) are disposed in supply component 10. Cell vessel 10a is a vessel holding blood (the first liquid) that includes hematopoietic stem cells (the first cells). Magnetic bead vessel 10j is a vessel holding a liquid (a second liquid) that includes magnetic beads for separating the hematopoietic stem cells from the blood. Viral vector vessel 10g is a vessel holding a liquid (a third liquid) that includes a viral vector for causing the hematopoietic stem cells (the first cells) to be infected with a virus. Separation vessel 20a is disposed in separation component 20. Separation vessel 20a holds: the blood (the first liquid) fed from cell vessel 10a (the first vessel); and the liquid (the second liquid) that contains the magnetic beads and is fed from magnetic bead vessel 10j (the second vessel). Infection vessel 30a is disposed in infection component 30. Infection vessel 30a holds the liquid that includes the hematopoietic stem cells (the first cells) and is fed from separation vessel 20a. Culture vessel 40a is disposed in culture component 40. Culture vessel 40a holds the liquid that includes the cells for which reprogramming has started (the second cells) and is fed from infection vessel 30a.

By feeding predetermined liquids from feeding origins to feeding destinations using these vessels, this configuration makes it possible to, using cell production device 1, automatically produce iPS cells.

Furthermore, in cell production device 1 according to the present embodiment, cell vessel 10a (the first vessel) and separation vessel 20a are linked by flow path 2, separation vessel 20a and infection vessel 30a are linked by flow path 2, and infection vessel 30a and culture vessel 40a are linked by flow path 2.

Since the respective vessels of supply component 10, separation component 20, infection component 30, and culture component 40 are thus connected by flow path 2, the iPS cells can be produced without being exposed to contaminants.

Furthermore, in cell production device 1 according to the present embodiment, supply component 10 includes: heat retention device 11a that performs heat retention of cell vessel 10a (the first vessel) and magnetic bead vessel 10j (the second vessel); and cold retention device 12a that performs cold retention of viral vector vessel 10g (the third vessel).

This configuration makes it possible to keep cell vessel 10a and magnetic bead vessel 10j at a constant high temperature and to keep viral vector vessel 10g at a constant low temperature, without being influenced by seasonal or daily temperature fluctuations or by temperature fluctuations due to the environment in which cell production device 1 is installed. This makes it possible to stabilize and unify the activity of the materials and the reagents.

Furthermore, in cell production device 1 according to the present embodiment, separation-use culture medium vessel 10c (a first culture medium vessel), infection-use culture medium vessel 10e (a second culture medium vessel), and culture-use culture medium vessel 10h (a third culture medium vessel) are disposed in supply component 10. Separation-use culture medium vessel 10c is a vessel holding a culture medium to be supplied to separation vessel 20a. Infection-use culture medium vessel 10e is a vessel holding a culture medium to be supplied to infection vessel 30a. Culture-use culture medium vessel 10h is a vessel holding a culture medium to be supplied to culture vessel 40a. Heat retention device 11a performs heat retention of separation-use culture medium vessel 10c and infection-use culture medium vessel 10e, and cold retention device 12a performs cold retention of culture-use culture medium vessel 10h.

By performing heat retention or cold retention according to the usage purpose, even for the same culture medium, this configuration makes it possible to supply, to a predetermined vessel, the culture medium at an appropriate temperature suited to that purpose. This makes it possible to efficiently produce iPS cells.

Furthermore, in cell production device 1 according to the present embodiment, supply component 10 is located higher than separation component 20 and infection component 30.

Thus disposing supply component 10, which serves as the feeding origin for liquid, vertically higher than separation component 20 and infection component 30, which serve as feeding destinations for liquid, makes it possible to establish a height difference between the feeding origins and the feeding destinations. This makes it possible to facilitate the feeding of liquid in accordance with Bernoulli's principle, as well as to mitigate pressure drops of the liquid in flow path 2.

Furthermore, cell production device 1 according to the present embodiment includes pressure-feeding component 70 that, by pressurization, feeds a liquid from supply component 10 to separation component 20 and infection component 30.

Liquids such as the materials, reagents, and the like used in producing iPS cells are present in a very small amount, but it is difficult to feed the very small amount of liquid in its entirety using only a tube pump. Accordingly, as in the present embodiment, feeding the liquid using pressurization instead of suction makes it possible to easily feed the liquid from supply component 10 to separation component 20 and infection component 30, even if the liquid is present in a very small amount. Moreover, in feeding a very small amount of liquid, feeding the liquid by pressurization makes it possible to inhibit contaminants from becoming mixed into the liquid (in other words, contamination), compared to a case of feeding the liquid by suction.

Furthermore, cell production device 1 according to the present embodiment includes: pressure gauge 5a that measures a pressure of the liquid fed by pressure-feeding component 70; and a determiner that determines that feeding of the liquid is completed, when the pressure measured by pressure gauge 5a decreases.

This configuration makes it possible to easily grasp, when a predetermined liquid in a liquid-holding vessel disposed in supply component 10 is fed to a vessel in separation component 20, infection component 30, or culture component 40, that the feeding of the total amount of the predetermined liquid is completed.

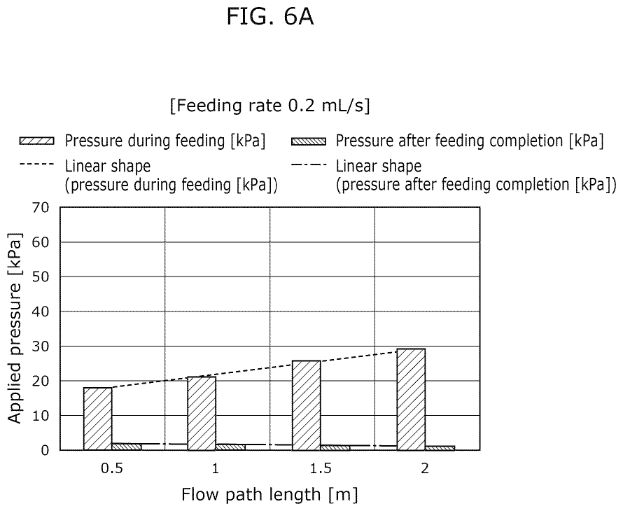

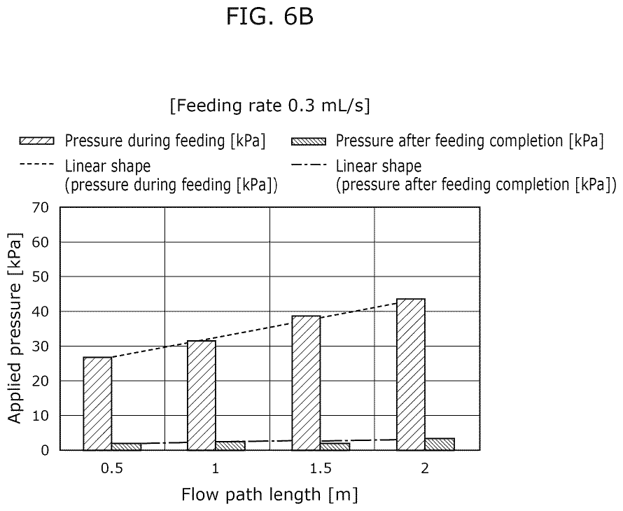

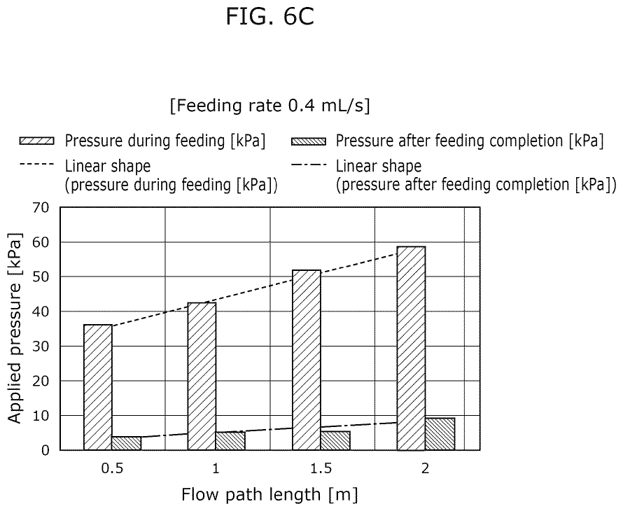

Here, an experiment into the relationship between the pressure while liquid is being fed (pressure during feeding) and the pressure after feeding of the liquid has been completed (pressure after feeding completion) was conducted. The results of this experiment will be described. Specifically, an experiment was conducted to measure the pressure difference between the pressure during feeding and the pressure after feeding completion, and to determine the dependence of this pressure difference on the flow path 2 length (flow path length) and on the liquid feeding flow rate (feeding rate). The results of this experiment are shown in FIG. 6A, FIG. 6B, and FIG. 6C. Each of FIG. 6A, FIG. 6B, and FIG. 6C shows the dependence of the pressure during feeding and the pressure after feeding completion on the flow path length. FIG. 6A illustrates a case in which the liquid feeding flow rate (feeding rate) is 0.2 ml/s, FIG. 6B illustrates a case in which the liquid feeding flow rate (feeding rate) is 0.3 ml/s, and FIG. 6C illustrates a case in which the liquid feeding flow rate (feeding rate) is 0.4 ml/s. It should be noted that in this experiment, as flow path 2 (the tube arrangement), a hose made of Teflon (registered trademark) having an inner diameter of 1.0 mm and an outer diameter of 1.6 mm was used. It should be noted that the same applies even if flow path 2 made of polyethylene is used.

As illustrated in FIG. 6A to FIG. 6C, it is revealed that when feeding of the liquid completes, the pressure decreases by one digit or more. Thus, when the pressure is measured by pressure gauge 5a and that pressure has dropped by one digit or more, it can be determined that the feeding is completed.

Furthermore, as illustrated in FIG. 6A to FIG. 6C, it is revealed that as the flow path length is greater, the difference between the pressure during feeding and the pressure after feeding completion is greater. In other words, when determining that the feeding is completed, flow path 2, which connects the feeding origin vessels and the feeding destination vessels, may be made as long as possible. For example, when causing the pressure after feeding completion to decrease by one digit or more, the length of flow path 2 is preferably set to 0.5 m or greater. This makes it possible to easily determine that the feeding is completed.

Furthermore, as seen by comparing FIG. 6A, FIG. 6B, and FIG. 6C, it is revealed that as the feeding flow rate is greater, the difference between the pressure during feeding and the pressure after feeding completion is greater. In other words, when determining that the feeding is completed, the feeding flow rate of the liquid fed through flow path 2 may be made as great as possible. For example, when causing the pressure after feeding completion to decrease by one digit or more, the liquid feeding flow rate is preferably 0.2 ml/s or greater. This makes it possible to even more easily determine that the feeding is completed. However, when the liquid feeding flow rate becomes too great, the liquid may foam up during feeding. Thus, the liquid feeding flow rate is preferably 0.4 ml/s or less. This makes it possible to inhibit the generation of foamy liquid during feeding.

### Embodiment 2

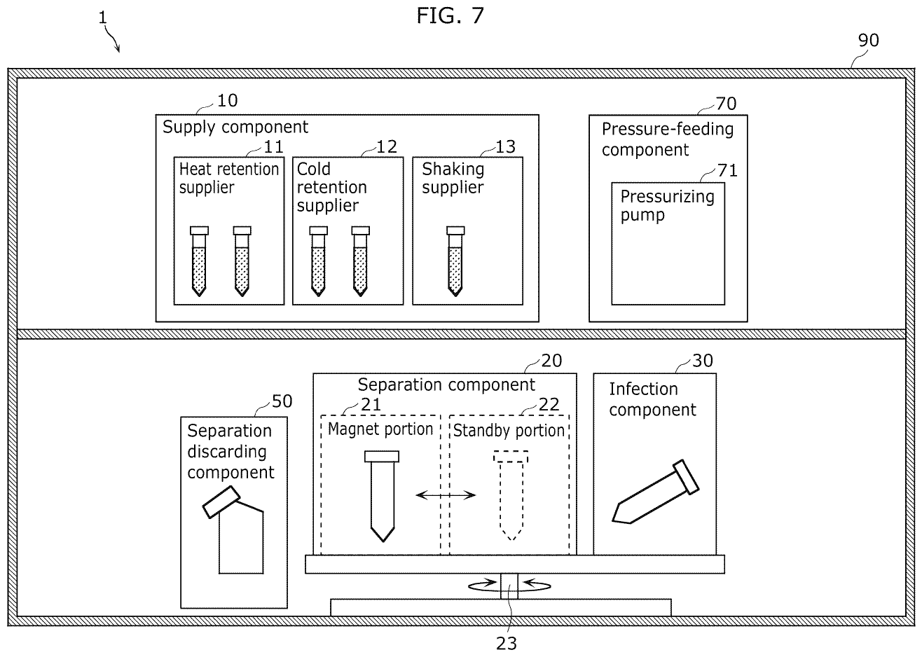

Next, Embodiment 2 is described. In Embodiment 1, cell production device 1 included: supply component 10 that supplies various reagents, materials, and a liquid that includes the cells serving as the basis for the target cells; separation component 20 that extracts and separates the cells from the liquid that includes the cells and was supplied from supply component 10; infection component 30 that causes the cells separated in separation component 20 to be infected to generate cells for which reprogramming has started; and culture component 40 that generates the target cells by culturing the cells for which reprogramming has started, but this is not intended to be limiting. In Embodiment 2, as illustrated in FIG. 7, cell production device 1 may include: supply component 10; separation component 20; and infection component 30. Since supply component 10, separation component 20, and infection component 30 are thus connected by flow path 2, iPS cells can be produced without being exposed to contaminants.

Specifically, cell production device 1 is a device that can produce infected cells. In the present embodiment, the target cells are cells for which reprogramming has started. Thus, cell production device 1 produces cells for which reprogramming has started. Infected cells are produced based on hematopoietic stem cells included in blood. Specifically, it is possible to produce cells for which reprogramming has started by: infecting hematopoietic stem cells extracted from the blood with a virus by using a viral vector; and introducing iPS genes into the hematopoietic stem cells. Therefore, cell production device 1 includes: a mechanism for extracting and separating, from blood, hematopoietic stem cells that serve as the basis for iPS cells; and a mechanism for imparting iPS genes to the extracted hematopoietic stem cells, and cell production device 1 is capable of automatically and continuously performing a series of steps until producing, from blood, cells for which reprogramming has started. It should be noted that in the present embodiment, the target cells may be iPS cells, and the cells for which reprogramming has started in infection component 30 may be iPS cells.

It is to be noted that the target cells produced in infection component 30 may be cultured by a culture device different from cell production device 1, to produce desired cells.

Furthermore, single-use kit 100 includes: the liquid-holding vessels disposed in supply component 10 (cell vessel 10a, PBS vessel 10b, separation-use culture medium vessel 10c, detachment liquid vessel 10d, infection-use culture medium vessel 10e, coating solution vessel 10f, viral vector vessel 10g, culture-use culture medium vessel 10 h, sterile water vessel 10i, and magnetic bead vessel 10j); separation vessel 20a disposed in separation component 20; infection vessel 30a disposed in infection component 30; discard vessel 50a disposed in separation discarding component 50; and flow path 2 that links these vessels. These vessels and flow path 2 are sterilized in advance.

### Variations

The cell production device and the cell production method according to the present disclosure were thus described above based on Embodiments 1 and 2, but the present disclosure is not intended to be limited to Embodiments 1 and 2.

For example, in Embodiments 1 and 2, the target cells were iPS cells or cells for which reprogramming has started, but the target cells are not limited thereto. Specifically, T cells obtained by inducing further differentiation of the produced iPS cells may be the target cells. In this case, cell production device 1 may include a mechanism that is able to induce differentiation of the produced iPS cells. Furthermore, the target cells may be stem cells other than iPS cells, such as ES cells, or may be cells other than stem cells.

Furthermore, in Embodiments 1 and 2, the iPS cells were produced from hematopoietic stem cells by using a viral vector, but this is not intended to be limiting. In other words, the iPS cells may be produced from hematopoietic stem cells without using a viral vector.

Furthermore, in Embodiments 1 and 2, the plurality of vessels used in cell production device 1 were a part of the replaceable single-use kit, but this is not intended to be limiting. In other words, these vessels need not be replaceable. For example, these vessels may be provided integrally with cell production device 1, and removal from cell production device 1 need not be possible. It should be noted that it is not necessary for all of the plurality of vessels to be replaceable. Some of the plurality of vessels may be replaceable.

Furthermore, in Embodiments 1 and 2, flow path 2 in cell production device 1 was a part of the replaceable single-use kit, but this is not intended to be limiting. In other words, the segments of flow path 2 need not be replaceable. For example, flow path 2 may be provided integrally with cell production device 1, and removal from cell production device 1 need not be possible. It should be noted that it is not necessary for all segments of flow path 2 to be replaceable. Some of the segments of flow path 2 may be replaceable. Furthermore, flow path 2 may be a part of vessel(s).

Furthermore, in Embodiments 1 and 2, the plurality of vessels used in cell production device 1 were connected by flow path 2, but this is not intended to be limiting. In other words, these vessels may be connected by connectors for connecting flow path 2 and these vessels together.

Furthermore, in Embodiments 1 and 2, when feeding liquid, the liquid was fed by applying pressure, but this is not intended to be limiting. For example, the liquid may be fed by suctioning in which a vacuum pump or the like is used.

The present disclosure also includes other forms obtained by making various modifications to Embodiments 1 and 2 that can be conceived by those skilled in the art, as well as forms obtained by combining constituent elements and functions of Embodiments 1 and 2 as desired, within a scope not departing from the spirit of the present disclosure. Furthermore, the present disclosure also includes all combinations of two or more claims, selected from among the plurality of claims recited in the claims of the present application at the time of filing, to the extent that such combinations are not technically inconsistent. For example, when the dependent claims recited in the claims at the time of filing the present application are rewritten as multiple dependent claims or as multiple-multiple dependent claims (i.e., multiple dependent claims that refer to another multiple dependent claim) by referring to all technically consistent higher-level claims, all combinations of the claims encompassed by such multiple dependent claims and multiple-multiple dependent claims are also included in the present disclosure.

### INDUSTRIAL APPLICABILITY

The techniques of the present disclosure are useful as a cell production device or the like for producing target cells such as iPS cells.

1. A cell production device comprising:
a supply component that supplies a first liquid that includes a first cell;
a separation component that separates the first cell from the first liquid supplied from the supply component; and
an infection component that causes the first cell separated in the separation component to be infected with a virus to generate a second cell, wherein
the supply component, the separation component, and the infection component are connected by a flow path.

2. The cell production device according to claim 1, wherein
a first vessel, a second vessel, and a third vessel are disposed the supply component, the first vessel holding the first liquid that includes the first cell, the second vessel holding a second liquid that includes a magnetic bead for separating the first cell from the first liquid, the third vessel holding a third liquid that includes a viral vector for causing the first cell to be infected with the virus,
a separation vessel is disposed in the separation component, the separation vessel being a vessel that holds the first liquid fed from the first vessel and the second liquid fed from the second vessel, and
an infection vessel is disposed in the infection component, the infection vessel being a vessel that holds a liquid that includes the first cell and is fed from the separation vessel.

3. The cell production device according to claim 2, wherein
the first vessel and the separation vessel are linked by the flow path, and
the separation vessel and the infection vessel are linked by the flow path.

4. The cell production device according to claim 2, further comprising:
a culture component that cultures the second cell generated in the infection component, wherein
the flow path connects the supply component, the separation component, the infection component, and the culture component.

5. The cell production device according to claim 4, wherein
a culture vessel is disposed in the culture component, the culture vessel being a vessel that holds a liquid that includes the second cell and is fed from the infection vessel.

6. The cell production device according to claim 5, wherein
the infection vessel and the culture vessel are linked by the flow path.

7. The cell production device according to claim 5, wherein
the supply component includes:
a heat retention device that performs heat retention of the first vessel and the second vessel; and
a cold retention device that performs cold retention of the third vessel.

8. The cell production device according to claim 7, wherein
a first culture medium vessel, a second culture medium vessel, and a third culture medium vessel are disposed in the supply component, the first culture medium vessel holding a culture medium to be supplied to the separation vessel, the second culture medium vessel holding a culture medium to be supplied to the infection vessel, the third culture medium vessel holding a culture medium to be supplied to the culture vessel,
the heat retention device performs heat retention of the first culture medium vessel and the second culture medium vessel, and
the cold retention device performs cold retention of the third culture medium vessel.

9. The cell production device according to claim 5, wherein
an air hole is provided in each of the separation vessel, the infection vessel, and the culture vessel.

10. The cell production device according to claim 1, wherein
the supply component is located higher than the separation component and the infection component.

11. The cell production device according to claim 1, comprising:
a pressure-feeding component that, by pressurization, feeds a liquid from the supply component to the separation component and the infection component.

12. The cell production device according to claim 11, comprising:
a pressure gauge that measures a pressure of the liquid fed by the pressure-feeding component; and
a determiner that determines that feeding of the liquid is completed, when the pressure measured by the pressure gauge decreases.

13. The cell production device according to claim 1, wherein
an inner diameter of the flow path is 0.5 mm or greater and 5 mm or less.

14. The cell production device according to claim 1, wherein
the supply component serves as a feeding origin for liquid,
the separation component and the infection component each serve as a feeding origin for liquid or a feeding destination for liquid, and
all valves not provided in the flow path between a feeding origin and a feeding destination are closed.

15. The cell production device according to claim 1, wherein
the first cell is a hematopoietic stem cell,
the first liquid is blood, and
the second cell is a cell for which reprogramming has started.

16. A vessel disposed in the cell production device according to claim 1.

17. A single-use kit that is replaceable and is used in a cell production device that produces a cell, the single-use kit comprising:
a liquid-holding vessel from which a first liquid that includes a first cell is supplied;
a separation vessel in which the first cell is separated from the first liquid supplied from the liquid-holding vessel; and
an infection vessel in which the first cell separated in the separation vessel is caused to be infected with a virus to generate a second cell, wherein
the liquid-holding vessel, the separation vessel, and the infection vessel are connected by a flow path.
